(19) United States
(12) Patent Application Publication
(10) Pub. No.: US 2012/0065562 A1
(43) Pub. Date: Mar. 15, 2012

(54) ORTHOTIC WITH LIMITED MIGRATION

(76) Inventor: Wieland Kaphingst, Lewistown, NY (US)

(21) Appl. No.: 13/321,508

(22) PCT Filed: May 19, 2010
(86) PCT No.: PCT/EP10/03053

§ 371 Date: Nov. 18, 2011

(30) Foreign Application Priority Data
May 20, 2009 (DE) 102009023129.3

(51) Int. Cl. A61F 5/01

(52) U.S. Cl. 602/12

(57) ABSTRACT

The invention relates to the technical field of orthotic appliances for the modification or stabilization of musculoligamentous and/or skeletal structures of the human or animal body, especially for stabilizing joints. The invention provides means for preventing the migration of the orthotic appliance applied to the body using a rigid frame element. The approach according to the present invention provides for the orthotic appliance to be secured on at least one anatomical protrusion on the body using a rigid frame element in a force-locking and form-fitting manner.

### BACKGROUND

The invention relates to the technical field of orthotics for the modification or stabilization of musculoligamentous and/or skeletal structures of the human or animal body, in particular for stabilizing the joints of the body. The invention provides means for preventing the migration of an orthotic appliance attached to the body using a rigid frame element. The approach pursued with the present invention provides for the orthotic appliance with a rigid frame element to be secured on at least one anatomical protrusion on the body in a force-locking and form-fitting manner.

### STATE OF THE ART

Orthotics are medical aids, which act therapeutically or prophylactically and serve primarily to stabilize a joint in the body and/or the motor system of the human or animal body. Orthotics are manufactured in various embodiments in accordance with the physiological or biomechanical task: so-called prophylactic orthotics serve primarily to prevent an injury to the motor system in situations of above-average physical stress, especially when there is a risk of overloading a joint of the body, especially in conjunction with athletic activity or extreme or dangerous physical exertion. They are characterized primarily by rigid frame structures and long lever arms, which have a protective effect by absorbing forces and torques acting on the joint, for example, the knee joint, and/or diverting them to structures distally from the joint (in the case of the knee joint, these would be the thigh and calf) so that the action of force on the joint to be protected is reduced.

So-called rehabilitative orthotics are used primarily postoperatively and especially also temporarily to preserve the result of the operation and to support healing or convalescence. Rehabilitative orthotics are subjected to lower forces and torques than the prophylactic orthotics. They require a mechanically less stable or rigid structure. Their technical embodiment may be simpler because it is associated with a shorter therapeutic use time. This simplification of the structure is not consistent with long-term use.

So-called functional orthotics are used in both the short-term postoperatively and in the long-term to reduce the danger to a joint on which surgery has been performed.

So-called pressure-relieving orthotics relieve the medial or lateral compartment of the joint due to their axial alignment or due to installed restraints or pressure elements by redistributing the load, in particular the force, and the pressure per unit of area to the other compartment respectively of the articulating joint surfaces.

In addition to this functional classification, orthotics are also classified according to their primary structural features: the category of so-called “rigid frame or rigid shell orthotics” (English “rigid orthotics”) comprises primarily orthotics which surround and cover the respective body part or joint in particular with a so-called shell or rigid shell over the surface area (metal or plastic component shaped to conform to the surface). Alternatively, the body part is surrounded by a force- and torque-strategic frame structure. Since the materials used for constructing the shell or frame are rigid, they must be arranged between the shell or frame components arranged above and below the joint [with] articulated or elastic connections to maintain the mobility of the joint. They are typically used as prophylactic and functional orthotics.

The category of so-called “soft good orthotics” (also known as and used interchangeably with “orthopedic soft goods” or “soft orthotics”) comprises orthotics which are embodied like bandages primarily made of textiles or flexible woven, cellular or noncellular plastics and are usually designed as orthotics that are tubular or otherwise surrounding the body part (e.g., knee joint) at the surface. They are cut from flat flexible plastics mainly by cutting and joining methods, or they may be knitted to shape from textile fibers with or without elastic components or cast in molds from castable elastomers. In some embodiments, they consist only of truss pads (pressure surfaces) which act on the body part and the fasteners for same. In other embodiments, they consist of soft materials which are strategically reinforced and are optionally additionally upgraded with tension belts and/or joints to fulfill certain anatomical/physiological functions. They are not usually assembled with joints because they can follow the movement of the joint due to their flexibility. These structures are not usually classified as orthotics in the narrower sense because they usually have only a minor effect biomechanically. They are typically used as compression orthotics and/or proprioceptive orthotics. With additional reinforcing elements as equipment, they increasingly extend into the category of so-called “hybrid orthotics.”

The category of so-called “hybrid orthotics” (English “semirigid orthotics”) comprises another group of orthotics which include both flexible elements, especially textile elements (such as a soft good orthotic) as well as rigid elements (such as a rigid frame orthotic), the latter primarily determining the function. Function-determining rigid elements include in particular side splints, which are optionally joined together with articulated joints or nonelastic textile components such as restraints. Hybrid orthotics are not typically upgraded with a complete rigid shell or a complete rigid frame surrounding the body. They are usually used as “rehabilitative orthotics.”

The function and use of orthotics will be explained below in the example of the orthotic for the knee joint. Knee braces serve primarily or entirely to provide physiological axial guidance of the knee joint, control of therapeutically indicated limitations of movements and torques (in extension and flexion of the knee joint), control of bending moments in the frontal plane (in the sense of control of nonphysiological varus or valgus moments), control of torsion moments between the femur and tibia (in the sense of rotation between the thigh and the calf about the longitudinal axis of the leg), partially relieving the stress on joint components (in the sense of redistributing the load on the joint surfaces), preventing the “anterior pivot shift” (e.g., a drawer-type movement of the head of the tibia forward=“anterior drawer” or “anterior drawer effect”) after rupture of the anterior cruciate ligament, preventing the “posterior pivot shift” (e.g., a drawer-type movement of the head of the tibia toward the rear=“posterior drawer” or “posterior drawer effect”) after rupture of the posterior cruciate ligament, absorbing impact and ground reaction torques which occur due to an external influence (impact collision in athletic processes) and can lead to the aforementioned forces and torques on the knee joint, the prophylactic or therapeutic compression of edema (soft tissue swelling) as well as the compression of soft tissue components to support the physiological proprioceptive spatial classification of the joint being treated (preventing injury).

To ensure these functions permanently, i.e., over the entire wearing time after the orthotic is placed on the body part or joint of a user, the orthotic must always remain in the location intended physiologically and in the three-dimensional arrangement of its function elements as intended with respect to the body part or joint. Otherwise its functioning is no longer ensured. Furthermore, an improperly placed orthotic may cause additional stress in the ligamentary and cartilage structure of a joint. Slippage or twisting of the orthotic during use, so-called migration, should be prevented or largely reduced in extent. Furthermore, applying a simple and secure positioning of the orthotic to the relevant physiological position (function position) in a manner that is simple for the user should be made possible. The state of the art does not contain any satisfactory approaches for preventing this problem.

With known orthotics, inadequate contact, i.e., inadequate adaptation to the anatomy of the user and the associated shortcomings in the transmission of torque and force are the main causes of unwanted slippage or twisting during use. This involves inadequate form-fitting and force-locking connection between the orthotic and the body part or joint (inadequate adaptation). This is also due in particular to an inadequate adherence to the soft tissue and the lack of congruence of anatomical and technical joint axes.

So far the following measures are known for overcoming these inadequacies: individual tailoring, taking into account all anatomically shaped features of the intended user; thermoplastically or otherwise reshapable shells, frames and splints; extensive adjustability in size, circumference and/or angles; adhesion promoters between the body part and the orthotic; polycentric joint splints or those which automatically find the anatomical pivot point.

One disadvantage is that these measures are technically complex, make the orthotic more difficult to handle and may impair the physiological function of the orthotic and its wearing comfort and nevertheless reduce migration only inadequately and/or unreliably, i.e., depending on the respective stress or movement situation.

### SUMMARY OF THE INVENTION(S)

The invention is based on the technical problem of further developing a known orthotic so that an unwanted migration of the orthotic when worn is reliably and adequately prevented or expediently reduced. Furthermore, the technical problem is based on reliably enabling the positioning of the orthotic in its function position in application and especially making its use simple for the user.

The technical problem is solved according to the invention by providing an orthotic or an orthotic part (10), which has at least one rigid frame element (20, 200) distally from the body, wherein an orthotic or orthotic part (10) is characterized primarily in that it has at least one coupling element (40), in particular elastic, proximally to the body in particular, i.e., facing the body, and the at least one rigid frame element (20, 200) of the orthotic or of the orthotic part has at least one recess (30) on the side facing the body, in particular in the form of a concave recess or cavity or fillet, which is suitable and is preferably designed specifically to receive the coupling element (40) in particular in a force-locking and/or form-fitting manner. According to the invention, the rigid frame element (20, 200) can be brought into force-locking and in particular additional form-fitting engagement with at least one anatomical protrusion (50) on the body, in particular a bony or cartilaginous protrusion over the recess (30) and over the coupling element (40), and the orthotic (10) can be affixed to the body in a stationary manner in this way by means of the coupling element (40) and the rigid frame element (20, 200).

The invention thus proposes that an improved orthotic having a rigid frame element or an improved orthotic part having a rigid frame element be provided, wherein at least one rigid frame element can be coupled directly to an anatomical protrusion on the body via at least one coupling element, preferably made of an elastomer, so that the orthotic or the orthotic part is affixed on the body and its unwanted migration on the body during use can thereby be prevented. The invention thus solves the technical problem on which it is based.

The form and in particular additional force-locking coupling of the orthotic to at least one body protrusion allows absorption of the transverse forces and transverse torques which occur during use and would otherwise cause migration of the orthotic out of its function position. To transmit these transverse forces from the orthotic to the body protrusion, the invention proposes in particular an elastic coupling element which allows the force-locking and preferably form-fitting coupling at least between the rigid frame element of the orthotic according to the invention and the body protrusion.

The targeted gripping of entire protrusions with form-fitting coupling elements, which are in turn surrounded in a form-fitting and force-locking manner by the rigid frame element of the orthotic designed according to the invention and are optionally also compressed, has not previously been known for orthotics having rigid frame elements or shells. This effect, which secures the adhesion and migration-free anchoring of a soft good orthotic that is preferably underneath it and forms a hybrid orthotic together with the rigid frame element, at the same time secures, according to the invention, the rigid frame or shell orthotic designed according to the invention and preferably attached to the top side of the orthotic in the function position provided therapeutically.

The design of the rigid frame element of the orthotic according to the invention provides at least one so-called “recess” to connect the coupling element according to the invention to the rigid frame element in a force-locking manner. For form-fitting accommodation of the coupling element, the recess is preferably designed as a concave indentation, in particular in the form of a cup or a pan. This type of recess preferably has a circular cross section, or as an alternative an elliptical cross section. The shape of the extent [sic; recess] is especially preferably adapted to the shape of the coupling element, especially the side facing the rigid frame element. The concave recess is preferably fenestrated at the center.

In an alternative or preferably an additional implementation of the force-locking coupling between the rigid frame element of the orthotic and the coupling element, at least one additional connecting element is provided between the coupling element and the rigid frame element. This connecting element is preferably designed as pins, hooks, buttons, grooves, serrations, loops, clamps or coupling by means of magnets or in a similar form which can be engaged with its respective counterpart on a coupling element or rigid frame element in a force-locking engagement. In an exemplary and preferred embodiment, a bolt is formed on the side of the coupling element pointing toward the rigid frame element, this bolt being engaged with a similarly dimensioned fenestration (hole) in the rigid frame element.

In a preferred embodiment, the force-locking coupling between the rigid frame element and the coupling element is accomplished by magnets which are preferably incorporated into the rigid frame element and by metal surfaces which are preferably arranged in or on the coupling element.

In another alternative, the connecting element, preferably in addition, is designed as an adhesive bond. The adhesive bond is preferably provided as a permanent connection between the rigid frame element and the coupling element. In one variant of this the adhesive bond is releasable. Those skilled in the art are familiar with suitable adhesives for this purpose. The term “recess” as used according to the invention to accommodate the coupling element and the rigid frame element is thus understood in the sense of this invention to also include the design thereof in the form of such a connecting structure, preferably in addition, or a part thereof.

In the case of an anatomical protrusion on the body which can be used according to the invention, preferably at least one bony or cartilaginous or muscular and usually cup-shaped convex structure is visually visible on the body, for example, the ankle, or is at least palpable, for example, the joint of the wrist. This anatomical protrusion is suitable for absorbing transverse forces and moments. It is self-evident that the anatomical protrusion may be designed with varying degrees of prominence depending on the physiological condition of the human or animal body, the quantity of fatty tissue and connective tissue covering the bony protrusion. The coupling element is preferably adapted to that. Anatomical structures which may of course have less of a fatty tissue covering are especially preferred.

A preferred material for constructing the coupling element preferably has two properties: (1) it is elastic to enable form-fitting coupling with the protrusion on the body but at the same time to avoid pressure spots on the body; (2) it develops a high coefficient of static friction in combination with the body surface, in particular skin or skin covered with hair or fur, so that high transverse forces on the protrusion on the body can be absorbed even when the contact pressure is low to prevent migration of the orthotic. Those skilled in the art will be familiar with materials suitable for this purpose in general from the field of orthotics. The coupling element preferably consists essentially, preferably exclusively, of an elastomer material or a composite material, in which case the material or materials are preferably selected from the group consisting of silicone elastomers, polyurethane elastomers, thermoplastic elastomers and elastic polymer foams.

To improve the adhesive friction between the coupling element and the body surface in the area of the anatomical protrusion, the invention preferably additionally provides a coating which promotes adhesion. Those skilled in the art will be familiar with materials and measures suitable for this purpose in general.

In a preferred alternative embodiment, an intermediate layer in particular in the form of a textile element such as terrycloth is provided between the coupling element and the body surface (skin). By preventing the elastomer material of the coupling element from coming into direct contact with the skin surface, wearing comfort can be improved, depending on the concrete application and embodiment of the orthotic. Due to the shaping of the coupling element over the anatomical protrusion to be coupled, an adequate force-locking and form-fitting coupling can nevertheless be created.

It is self-evident that the concrete form and embodiment of the coupling element is or may also be adapted to the use requirements of the orthotic in wearing, applying and/or depositing as well as in particular the concrete shape of the anatomical protrusion. In one embodiment the coupling element is specifically designed to reach around the at least one anatomical protrusion in the form of a ring, a half ring or a bracket. Accordingly the coupling element is preferably designed in the form of a ring, essentially having the shape of a ring-shaped torus, an ellipsoidal or oval torus.

In an alternative embodiment, the coupling element is not designed in the form of a closed ring body but instead as a ring body, which is open on one side, in particular in the form of a semicircle or a horseshoe.

In an alternative embodiment, the coupling element is essentially disk-shaped, especially circular-disk-shaped or ellipsoidal, in particular in the form of an oblate rotational ellipsoid, or is oval or egg-shaped, in particular in the form of an oblate rotational ellipsoid (English “spheroid”) or some other ellipsoid. These basic shapes may also be designed to lie side by side, in particular in the form of a Figure eight (two ring-shaped structures) or a ring chain. In a preferred variant of this embodiment, the coupling element has a material thinning (reduced material thickness), recess or fenestration preferably at the center to enable or facilitate the centering of the coupling element over the anatomical protrusion.

The coupling element, preferably alternatively or additionally, has at least two zones with different values for the modulus of elasticity. These zones are preferably arranged concentrically, with the inner zone preferably having an increased yield in comparison with the anatomical protrusion, which improves the centering as well as the force-locking and form-fitting effect of the coupling element on the anatomical protrusion.

All the embodiments outlined briefly above have in common the fact that the coupling element can be centered over the anatomical protrusion. The resulting improvement thereby achieved in the force-locking and form-fitting effect makes it possible for the coupling element to be able to absorb lateral forces (transverse forces) in particular acting parallel to the surface of the body from the orthotic, these forces being directed to the coupling element via the rigid frame element designed according to the invention, and in doing so it may remain secured over the anatomical protrusion essentially in an unchanging location. Centering of the coupling element over the protrusion allows, first of all, an improvement in the force-locking effect between the coupling element and the protrusion to absorb the transverse forces of the orthotic. Secondly, and due to the centering which is preferably provided, a more accurate or simplified positioning of the orthotic is made possible. Therefore, other measures which are provided for positioning purposes with traditional orthotics may be omitted, such as a targeted belt guidance of the belt to secure the position. The inventive measure for positioning the orthotic allows an alternative method of belt guidance which is more advantageous for the wearer of the orthotic and in particular is more appropriate medically and/or is more comfortable.

To enable the force-locking and form-fitting effect of the coupling element with the anatomical protrusion as provided according to the invention, it is provided that the coupling element is pressed against the anatomical protrusion with a contact pressure which acts essentially perpendicular to the surface of the body in the direction of the anatomical protrusion. This contact pressure is exerted on the coupling element via the rigid frame element according to the invention, which has the recess, in particular the concave recess and/or fenestration to receive the coupling element.

The required contact pressure for coupling the coupling element to the anatomical protrusion as well as the rigid frame element to the coupling element is created either exclusively or additionally by the inherent elasticity of the rigid frame element, depending on the specific embodiment of the orthotic, as a function of the anatomy of the body part to which the orthotic is applied and the protrusion selected for fixation. The rigid frame element is preferably designed to be bracket-shaped or clamp-shaped in this preferred embodiment and produces a targeted pressure of the inventive coupling element against the at least one anatomical protrusion because it extends preferably completely around the body part to which the orthotic is to be secured.

The rigid frame element is therefore optionally operatively connected to at least one additional rigid frame element of the orthotic in which case the body part is surrounded at least to the extent that the contact pressure can be created on the side of the coupling element via a resulting force vector on the opposite side of the body part. In a preferred embodiment of the orthotic, this opposing force may also be created by an essentially opposite additional coupling element, which preferably acts on another anatomical protrusion, preferably an essentially opposite anatomical protrusion. In another variant, the opposing force is created by a large area support of the rigid frame element, which is optionally cushioned, at least on the opposite side.

In an alternative embodiment, a form-fitting clamping of the rigid frame element in the form of a partial enclosure with wing straps designed to form lateral rigid frame ends is provided for a form-fitting clamping of the body part, preferably in the form of a horseshoe-shaped clamp or bracket. These protrusions are especially preferably less rigid and are designed to be comparatively flexible, so as not to interfere with the movement of the bodily structures underneath, especially muscles, while at the same time exerting the restraining or clamping effect.

In an alternative or preferably additional embodiment the required contact pressure is created by a corresponding belt. An embodiment of the rigid frame element according to the invention therefore provides for this purpose at least one belt strap which can be guided around the body part and can be connected to the rigid frame element. Those skilled in the art can readily adapt the corresponding belt guides to the orthotic appliance as designed to create on the coupling element a contact pressure in the sense of the invention with a resulting force vector acting preferably perpendicularly on the body surface and the anatomical protrusion.

The following embodiments are especially preferred for the implementation of the invention as a knee brace: ring-shaped enclosure of the fibula head; flat enclosure of the anterior tibia edge and creation of an anchoring pin for locking on a concavity or recess in the rigid frame element; a cup anchoring which is pressed flat against muscle bulges with the superstructure of one anchoring journal for locking in a concavity or recess in the rigid frame element.

In another embodiment of the invention as a knee brace, the following embodiments are especially preferred: ring-shaped or horseshoe-shaped enclosure of the patella with the inventive coupling element, preferably designed as a so-called patella elastomer ring with a rigid frame element above it having a concave recess to receive the coupling element coupled to the patella. Toward the back of the knee, a belt is provided to press the rigid frame element with the coupling element against the patella and hold it there.

In a preferred embodiment of the recess, an impact protection shell is applied above the fenestration in the rigid frame element in the area of the coupling element. This impact protection shell is designed as follows: a cap of hard material resembling a segment of a sphere is placed on the coupling element (for example, a patellar elastomer ring) by means of an elastic belt or by means of anchoring joints at the side. The function of this cap is to protect the patella from the effect of an impact. It is constructed so that it is preferably in contact with the aforementioned elastomer ring on all sides. An impact in the direction of the patella is absorbed by this hard material cap and dissipated in the underlying elastomer element which forms an elastic buffer. The kneecap is so far beneath the protective device that it remains protected from impact.

The rigid frame element or rigid shell is designed in either one or more parts to form the orthotic appliance. A multipart design, in particular a two-part design, is preferred, where the parts are joined together in a force-locking manner preferably so that they are flexible but inelastic to form the orthotic appliance. Preferred designs of these embodiments involve at least two rigid frame elements or rigid shells preferably divided along the joint axis.

In a preferred embodiment, at least one rigid frame element according to the invention is connected with an articulated joint to at least one additional rigid frame element to form the inventive orthotic. The joints preferably provided for this purpose are typically monocentric or polycentric constructions. Polycentric joints which are better able to approximate the congruence of the natural knee joint fulcrum are especially preferred for the use of a knee brace. Biaxial joints optionally with forced coupling through articular splint teeth on the end face are most preferred. Instead of the front teeth, a friction wheel coupling may also be selected. In a preferred alternative embodiment, the articulated connection is worked homogeneously out of the orthotic material used for the appliance. The advantages of this embodiment include in particular the fact that it can be manufactured in a single operation. It does not require any arrangement or attachment of individual splints.

In a preferred embodiment, at least the rigid frame element according to the invention is embodied as a so-called rigid shell. In a first variant this rigid shell is designed so that it is adapted to the body part in a direct form-fitting manner and can be applied directly to it. In another more preferred variant the rigid shell is designed so that it can be attached in a form-fitting manner to a soft good orthotic appliance to form a hybrid orthotic.

In an alternative embodiment, the rigid frame element according to the invention is designed in one part or in one piece. This is especially preferred in conjunction with immobilizing orthotic appliances to stabilize an anatomical joint or to secure a body part in one position.

In a preferred embodiment, the rigid frame element according to the invention may preferably be applied from the outside to a soft good orthotic appliance which is preferably situated beneath it, i.e., toward the body (proximally to the body). The rigid frame element part together with the soft good orthotic appliance part preferably forms a hybrid orthotic. The subject matter of the present invention is thus also a hybrid orthotic appliance which has a soft good orthotic part which is preferably applied directly to the body, i.e., proximally to the body, at least one coupling element for coupling an anatomical protrusion and at least one rigid frame element part situated above it and more distally from the body such that the coupling element and the rigid frame element part are designed according to the present invention.

The coupling element is preferably arranged on or directly in connection with or inside the soft good orthotic appliance and together with it preferably forms a unit which is preferably inseparable. The coupling element is preferably connected to the soft good orthotic appliance in such a way that it is “applied” to the body part together with it. The migration-preventing effect achieved according to the present invention is then achieved when the rigid frame element designed according to the present invention is applied to the soft good orthotic appliance in such a way that the rigid frame element enters into a force-locking engagement with the coupling element of the soft good orthotic appliance arranged in the area of the anatomical protrusion over the recess provided according to the present invention, preferably in the form of a concave recess or a fenestration. The contact pressure on the coupling element of the soft good orthotic appliance is exerted via the applied or “finished” rigid frame element so that a force-locking and form-fitting “composite” of rigid frame element more distally from the body, soft good orthotic appliance proximally to the body and the coupling element in combination with the anatomical protrusion is formed, thereby preventing migration of the resulting hybrid orthotic appliance as a whole.

The invention thus provides an orthotic appliance or an orthotic part which is secured to the body over an anatomical protrusion. This is in contrast with other function principles of known orthotic appliances or orthotic parts, in which measures can be taken to secure or affix body elements, in particular musculoligamentous or skeletal structures in a certain position of the body to achieve a therapeutic or prophylactic result. Examples of this include the fixation of the patella on the knee joint, for example, when the physiological position of the patella is no longer ensured due to injury or degeneration. In this case, the orthotic appliance serves to reposition a body part. However, this body part does not serve to secure the orthotic. This would violate the therapeutic or prophylactic principle of an orthotic appliance. An inventive orthotic appliance in this case preferably has at least one additional structural design which is different from the inventive concave shaping of the rigid frame element according to the invention in conjunction with the inventive coupling element. This additional structural design serves the purpose of therapeutic and prophylactic fixation or repositioning of a body part. Thus the present invention provides in this embodiment for the orthotic appliance or the orthotic part (a) to be affixed to the body via at least one fixed anatomical protrusion and, optionally in addition, (b) another body part, preferably requiring treatment or protection, to be affixed preferably in its physiological position on the body or to be repositioned there optionally over a protrusion formed by this additional body part. The inventive orthotic appliance or the inventive orthotic part therefore has preferably at least one additional recess which is arranged on the rigid frame element according to the invention or on another rigid frame element such that the additional recess is specially designed for positioning, affixing or repositioning a musculoligamentous and/or skeletal structure on the body. This additional recess for therapeutic or prophylactic positioning of a body part is different from the first concave recess for affixing the orthotic appliance according to the invention. In a preferred embodiment, this additional concave recess is specially designed to receive an additional coupling element which enables the therapeutic and/or prophylactic fixation or repositioning of a body part in a form-fitting manner and in particular additionally in a force-locking manner.

In one exemplary embodiment, at least the coupling element which serves to affix the knee brace over the head of the tibia is applied to or incorporated into a soft good orthotic appliance applied directly to the body. For fixation according to the present invention, a rigid frame designed according to the invention is upgradable with a concave recess to receive the coupling element on the soft good orthotic appliance.

To permit the upgradability of the rigid frame element according to the invention on a soft good orthotic appliance as described above to yield a migration-free hybrid orthotic, it is preferably provided that at least one section of the distal rigid frame element, in particular on its inside facing the body, is adapted to the surface shape of the outside of the soft good orthotic appliance applied to the body in particular. The most proximal form-fitting coupling of the rigid frame element distally from the body and the soft good orthotic appliance, which is applied more proximally to the body, is preferably provided at least in this contact section. Thus, in this preferred embodiment, the rigid frame element according to the invention is designed so that it is not adapted directly to the surface of the body to which it is to be applied but instead is adapted to the soft good orthotic appliance with the coupling element arranged in between.

In preferred variants of embodiments, the rigid frame part is affixable or connectable to the soft good orthotic appliance by means of a releasable force-locking connection. The releasable connection is preferably selected from the group consisting of: belt, Velcro-type closure, screw connection, pin connection and button connection. The belt closure is implemented according to the invention in an essentially known manner.

Alternatively or additionally a Velcro-type connection is also provided. In a preferred embodiment, a loop and hook strip is glued preferably over the surface on the inside of a rigid frame element designed as a rigid shell. This closure then engages in the textile loops of the textile segment or elements attached to it (for example, the hook substrates for hook and loop closures) of the soft good orthotic appliance.

Alternatively or additionally a screw connection is provided. A preferred embodiment of this variant provides for multiple inside threads to be provided in a packet inside the structure, in particular the textile structure of the soft good orthotic appliance by plastic sheathing, by adhesive attachment or by ultrasonic welding. The screw is then anchored via the orthotic shell on the textile at the “anchor sites” formed by the inside thread in the soft good orthotic appliance by means of matching boreholes in the rigid frame element designed as an orthotic rigid shell.

Alternatively or additionally a pin connection is also provided. One or more stud bolts (pins) applied to the textile of the soft good orthotic appliance pass through matching boreholes in the rigid frame element. The rigid frame element can be affixed to the soft good orthotic appliance with sliders, cross pins, clamping disks, screw connections.

Alternatively or additionally a button connection is also provided. A compressible elastomer mushroom head (“bollard,” “cup button”) which is connected mechanically in a nonreleasable manner to the structure, in particular the textile structure, of the soft good orthotic appliance by casting can be buttoned/rebuttoned through a borehole in the rigid shell.

In a preferred embodiment, at least one shapable rigid frame wing is formed on the rigid frame element, in particular to enable an optimum adaptation to the shape of the soft good orthotic appliance underneath. Such “wings” can preferably be shaped thermoplastically and can be optimally adapted to the soft good element underneath by targeted heating and the application of force. Alternatively the wings may be made of a metallic material or a metallic composite material. Aluminum sheeting, optionally with layers of plastic applied to it, is preferred here. The sheets may be bent into the desired shape by applying force.

The hybrid orthotic appliance according to the invention yields the following advantages in particular, which are presented here for the case of a knee brace as an example. As long as the user of the knee brace is indoors in his household area and his knee joints are exposed to comparatively low forces and torques on the average, he will use the soft good orthotic appliance. In addition to the proprioceptive support of autonomous joint control on the part of the user and compressive detumescent therapy, this also offers some minor protection against forces and torques acting on the joint from the outside. However, as soon as the user of the knee brace is, for example, in athletic activity and his knee joints are subjected to comparatively high forces and torques, he can make use of the upgradable rigid frame construction for the creation of an individualized hybrid orthotic appliance which offers in particular mechanical control of the joint movement and the limitation of forces and torques which can have a negative effect on the knee joint in sports due to the naturally rigid construction. The rigid frame element is therefore adapted to the shape features of the soft good orthotic appliance and can be applied in a force-locking and form-fitting manner over the soft good orthotic appliance. The soft good orthotic appliance no longer needs to be removed from the user's leg for upgrading him with a rigid frame or shell construction. Conversely, the rigid frame or shell orthotic appliance can be removed by the user to return to the function of the soft good orthotic. The soft good orthotic may remain on the user's leg, so it need not be reapplied. In the field of sports in particular or in varying stress conditions, it is an essential advantage for the user to be able to switch rapidly and without any problems between two orthotic appliances of different function qualities and joint protection qualities.

The present invention also relates to the use of the rigid frame element characterized above having a recess to receive a coupling element in combination with the inventive coupling element for stationary fixation of an orthotic appliance on an anatomical protrusion on the human or animal body. The subject matter of the invention is in particular the use of a ring-shaped or half-ring-shaped or bracket-shaped preferably elastomer coupling element in combination with a rigid frame element according to the invention of an orthotic appliance for the stationary fixation of this orthotic appliance on an anatomical protrusion of the human or animal body. In the preferred embodiment, the coupling element is preferably elastic and is arranged near the body (proximally) and surrounds the anatomical protrusion in a force-locking manner and in particular in a form-fitting manner. The rigid frame element has a recess, preferably a concave indentation, to receive the coupling element.

The invention will now be described in greater detail by the following Figures and examples although these should not be understood to restrict it in any way. It is self-evident that one or more detailed approaches and features presented in the examples could also be applicable in conjunction with the embodiments described above without having to implement additional features or all the features described here.

### BRIEF DESCRIPTION OF THE DRAWINGS

FIG. 1A shows a side view of an orthotic appliance with a rigid frame element;

FIG. 1B shows a perspective view of the orthotic appliance of FIG. 1A;

FIG. 2A shows a side view of a knee joint;

FIG. 2B shows a frontal view of the tibia head of the calf;

FIG. 2C shows a side view of a knee joint;

FIG. 2D shows a frontal view of the tibia head of the calf;

FIG. 3A shows a side view of a soft orthotic appliance;

FIG. 3B shows a frontal view of the soft orthotic appliance of FIG. 3A;

FIG. 3C shows a side view of a rigid frame element with the soft orthotic appliance of FIG. 3A;

FIG. 3D shows a frontal view of a rigid frame element with the soft orthotic appliance of FIG. 3A;

FIG. 4A shows a frontal view of the coupling elements of the orthotic appliance of FIG. 1A in relation to the tibia head and patella;

FIGS. 4B through 4D show various embodiments of the orthotic appliance of FIG. 1A;

FIG. 5A shows a rear view of the coupling element of an orthotic appliance attached to the ankle of a foot;

FIG. 5B shows a rear view of an alternative embodiment of the orthotic appliance of FIG. 5A;

FIG. 5C shows a rear view of a foot orthotic appliance;

FIG. 5D shows a side view of the foot orthotic appliance of FIG. 5C.

### DETAILED DESCRIPTION

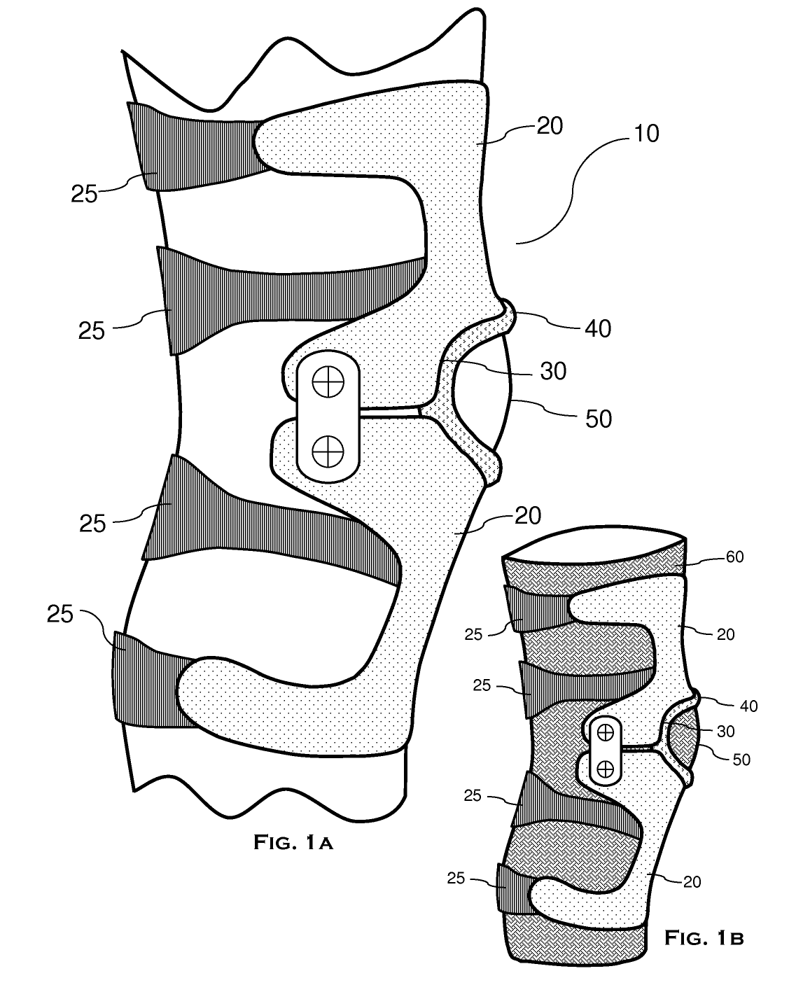

FIG. 1 shows an inventive orthotic appliance (10) for application to the knee joint, the appliance being composed of a rigid frame element (20) designed in two parts. The two rigid frame element parts are joined together with an articulation provided by a double joint. The rigid frame element forms a recess (30) to receive a coupling element (40) such that the rigid frame element (20) is brought into force-locking engagement with an anatomical protrusion (50), namely here the patella of the knee joint, over the recess (30) and the coupling element (40), to prevent migration of the orthotic appliance (10) on the knee. The rigid frame element (20) is applied to the leg in a force-locking manner via the belt system (25) spanning the leg. FIG. 1a shows a first specific embodiment of the inventive orthotic appliance (10). The coupling element is preferably attached securely directly to the rigid frame element and can be applied to the leg or removed from it together with this rigid frame element. In an alternative embodiment, which is illustrated in FIG. 1b, the rigid frame element (20) is part of the hybrid orthotic appliance which is formed from the rigid frame element (20) and a soft good orthotic appliance (60) applied to the leg and situated beneath the rigid frame element (20) which is also applied to the leg. It is optionally provided here that the coupling element is assigned directly to the soft good orthotic appliance (60) and is preferably securely attached to it. In this embodiment, the coupling element (40) together with the soft good orthotic (60) in the form of a stocking bandage is pulled onto the knee. To complete the inventive hybrid orthotic, the inventive rigid frame element (20) together with the recess (30) provided according to the invention may then be applied to the stocking bandage, in which case the rigid frame element (20) is brought to engagement with the anatomical protrusion (50), namely the patella of the knee joint here, over the recess (30) which is preferably embodied as a fenestration extending around the coupling element (40) in a force-locking and form-fitting manner, this in turn being connected in a force-locking and form-fitting manner, to secure the orthotic on the body in a stationary position. The rigid frame (20) according to the invention is preferably secured by means of a system of belts (25) to the knee joint and the underlying soft good orthotic (60), which is embodied here as a stocking bandage, underneath the soft good orthotic. In an alternative embodiment, the rigid frame element (20) is applied to the soft good orthotic (60) by other fastening means, in particular by Velcro-type closures, button closures, pin closures and the like; the belt closures (25) may then be omitted.

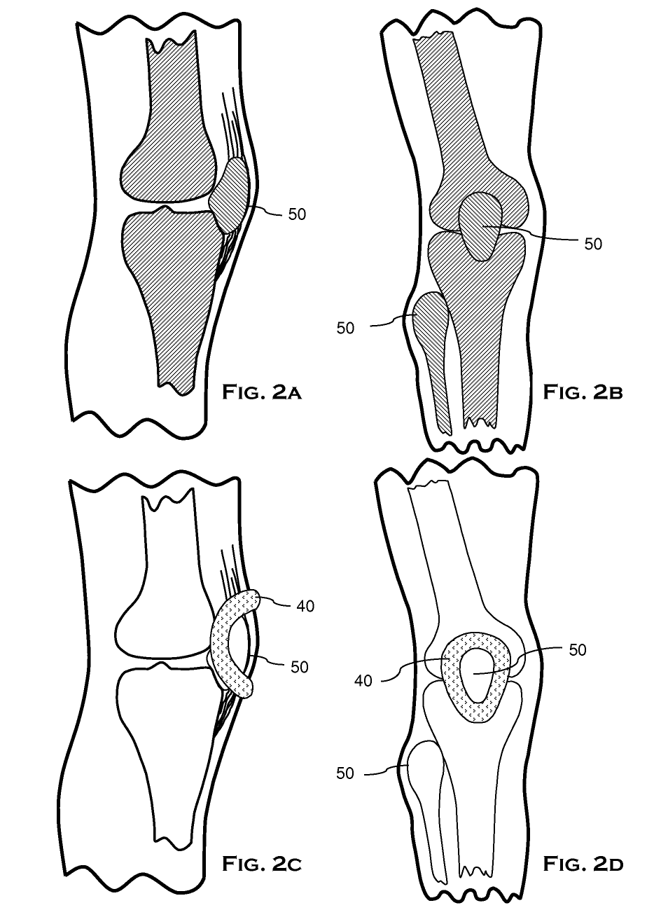

FIG. 2 illustrates the anatomical relationships of the body using the example of the knee joint with a prominent anatomical protrusion (50) formed by the patella. FIGS. 2a and 2c show the side view of the knee joint. Another protrusion (50) suitable for securing an orthotic appliance, is the tibia head of the calf, which is shown in a frontal view in FIGS. 2b and 2d. FIGS. 2c and 2d show the ring-shaped elastomer coupling element (40) as an example, which extends around the anatomical protrusion (50) of the patella in a form-fitting manner to affix the orthotic appliance (10) which is applied thereto and is formed with the rigid frame element (20) in a stationary position on the anatomical protrusion (50).

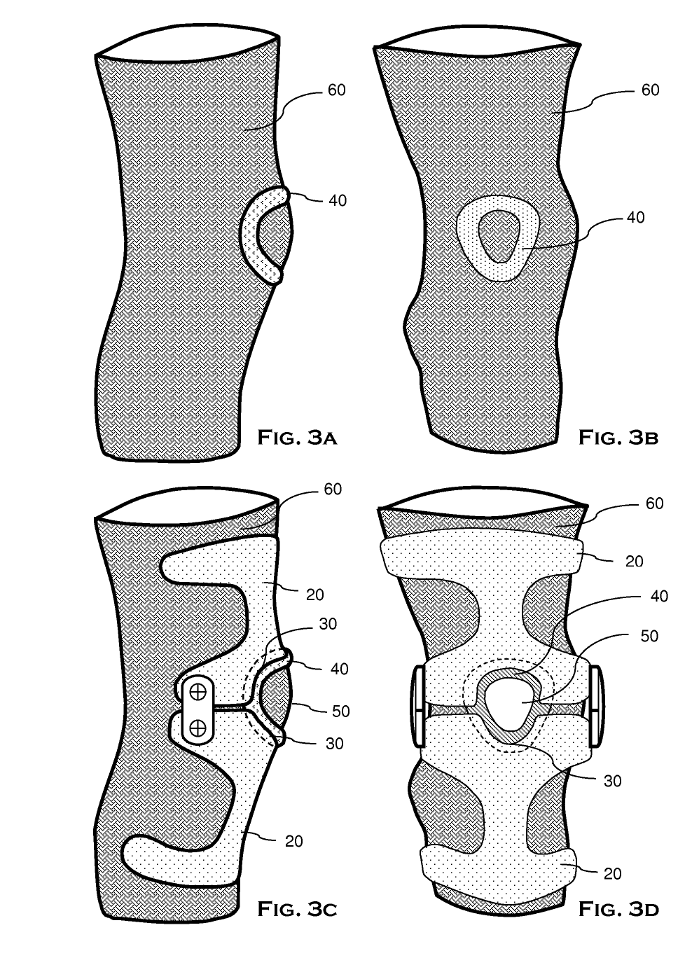

FIG. 3 shows a soft good orthotic appliance (60) designed as a stocking bandage with a ring-shaped coupling element (40) arranged in the anatomically correct position, i.e., above the anatomical protrusion (40) [sic; (50)]. In this embodiment the coupling element (40) is connected directly to the soft good orthotic (60) and is applied together with the latter to the anatomical joint (FIG. 3a, side view; FIG. 3d, frontal view). FIGS. 3c and 3d show the similar structures from FIGS. 3a and 3b with a rigid frame element (20) provided to form a hybrid orthotic appliance, having a recess (30) which is formed for the fenestration and which then extends around the coupling element (40) preferably fixedly mounted on the soft good orthotic to thereby secure the resulting orthotic (10) on the anatomical protrusion (50). The embodiment shown in FIGS. 3c and 3d preferably does not include belt systems; the rigid frame element here is secured on the soft good orthotic device (60) which is designed here as a fabric stocking secured by means of a Velcro-type closure, a button closure, a pin closure or the like.

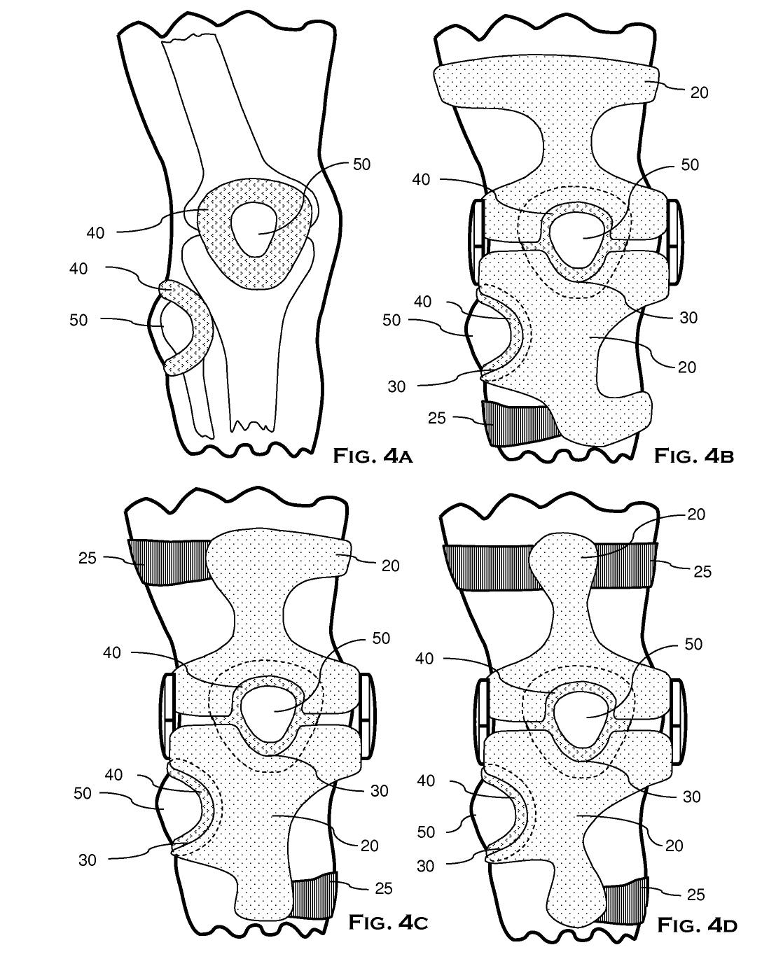

FIG. 4 shows additional embodiments of an inventive orthotic (10). FIG. 4a shows the anatomical relationships with the two anatomical protrusions (50), namely the tibia head and the patella, which are preferably gripped by the inventive coupling elements (40), preferably in the form of a ring or alternatively in the form of a clasp. FIGS. 4b, 4c and 4d each show alternative embodiments of a rigid frame (20) which is designed in two parts according to the present invention and has recesses (30) formed in the area of the anatomical protrusions (50) as a fenestration to secure the rigid frame element (20) on the anatomical protrusions (50) by means of the coupling elements (40). A system of belts (25) may optionally be provided.

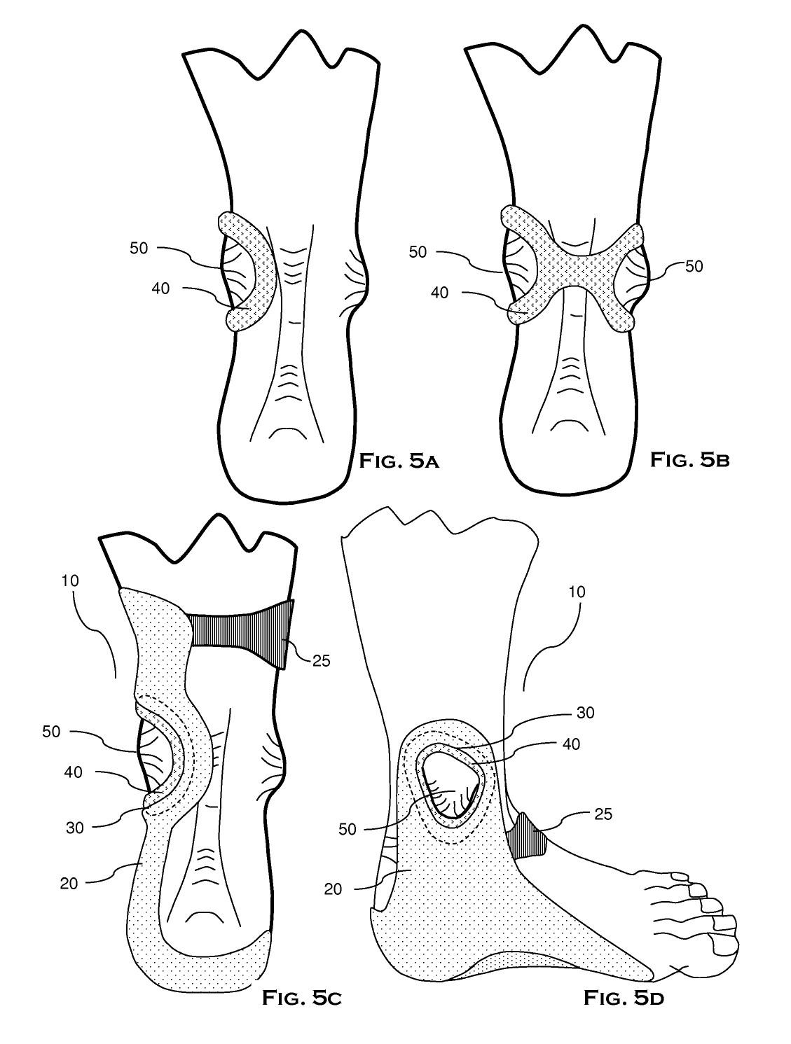

FIG. 5 shows another embodiment of the inventive orthotic (10) which is secured on the ankle of the foot. FIGS. 5a and 5b show preferred embodiments of the coupling element (40) which surrounds the ankle of the foot as the anatomical protrusion (50). FIG. 5a shows a coupling element (40) designed as a ring or a clasp. FIG. 5b shows a coupling element (40) which is designed in the form of a double ring or a Figure eight which here extends around the inner ankle as well as the outer ankle. A recess (not shown here) is preferably provided here on the coupling element (40) for the Achilles tendon. FIGS. 5c and 5d show alternative embodiments of a foot orthotic which is secured on the body in a stationary manner by means of the coupling element (40) using the ankle as the anatomical protrusion (50) selected according to the invention. To do so, the rigid frame element (20) provides a recess (30), which is designed as a fenestration and extends around the coupling element (40). The rigid frame element (20) is optionally secured on the foot additionally by at least one belt (25) which preferably extends around the foot or the leg.

The examples presented below all implement the principle of encompassing anatomical protrusions by means of coupling elements resembling an elastomer ring, which are in turn covered entirely or partially by a rigid frame orthotic or a shell orthotic.

### Example 1

### Cervical Orthotic Appliance Attached to the Chin

The cervical orthotic appliance contains as the inventive coupling element an appliance resembling an elastomer ring on the chin and is covered with an additional rigid shell as the inventive rigid frame element which additionally has a fenestration and sits on the chin in a form-fitting and force-locking manner. The rigid shell secured in this way offers a point of attachment for restraints (tension belts, struts, shells, etc.) of the orthotic appliance. These struts are mutually supported in the cervical area (cervical orthotic) or in the case of an embodiment as a thoracic cervical orthotic, they are secured in the upper thoracic area (cranial support).

### Example 2

### Occipitally Secured Thoracocervical Orthotic

As in Example 1, two elastomer rings or Figure-eight-shaped elastomer coupling elements are provided on the occipital bone of the skull for reaching around the tuberculosity of the occipital bone, which is covered with a rigid shell which optionally has a fenestration and sits there in a form-fitting and force-locking manner. The rigid frame element secured in this way serves as an attachment point for supports (struts, shells, etc.). To form a thoracocervical orthotic, struts which mutually support one another in the area of the back and shoulders and contribute toward relieving the burden on the cervical spine are provided.

### Example 3

### Cervically Fixed Cervical Orthotic

Like Example 1 or 2, an elastomer ring around the seventh cervical vertebrae (C7) is provided and is covered with a rigid shell which optionally has a fenestration and sits in a form-fitting and force-locking manner. This serves to provide an attachment point for restraints (tension belts, struts, shells, etc.). These struts act on the occipital bone in the cervical area and contribute toward relief on the cervical spine (cervical orthotic).

### Example 4

### Shoulder Orthotic Secured on the Acromion

An appliance resembling an elastomer ring for the acromion of the shoulder is provided. It is covered by a rigid shell which is optionally fenestrated and sits there in a form-fitting and force-locking manner. It serves to provide an attachment point for restraints (tension belts, struts, shells, etc.). Tension belts act mutually on a sleeve/strap/shell in the upper arm area and contribute toward relieving some of the stress on the shoulder due to the weight of the arm. In another embodiment, they serve to guide the ball of the head of the humerus in the socket of the joint and hold it there (shoulder-arm stress-relieving orthotic, shoulder-arm repositioning and guiding orthotic).

### Example 5

### Elbow Orthotic Secured on the Epicondyle

An appliance resembling an elastomer ring for one or both epicondyles is provided on the medial and lateral epicondyles of the humerus. Each one is covered with a rigid shell, optionally fenestrated, sitting in a form-fitting and force-locking manner. It serves to provide an attachment point for restraints (tension belts, struts, shells, etc.). Such restraints may act on a sleeve/strap/shell in the forearm area and contribute toward partially relieving the stress of the weight of the forearm on the elbow joint or guiding the articular axis and articular surfaces of the forearm (ulna) in the socket of the joint and holding it there (elbow relief and/or guidance orthotic).

### Example 6

### Wrist-Bone-Secured Metacarpal-Forearm Orthotic

An appliance resembling an elastomer ring is provided on at least one knuckle of the wrist or knuckle of the finger joints and is covered with an optionally fenestrated rigid shell sitting there in a form-fitting and force-locking manner. Restraints (tension belts, struts, shells, etc.) act on the rigid shell to contribute toward partially relieving the stress on anatomical structures (metacarpus, finger) situated further distally on one or more sleeves/straps/shells in the area of the hand or guiding the articular axis and articular surfaces thereof and holding them there (metacarpus or finger Quengel orthotic, stress relieving and/or guiding orthotic). An additional use is for supporting a metacarpal forearm orthotic appliance.

### Example 7

### Lumbar Orthotic Secured on the Spina Iliaca

A fixation attached to the anterior and/or superior spina iliaca on the pelvis is provided. An appliance resembling an elastomer ring gripping both spina iliacae is covered with a rigid shell, which is optionally additionally fenestrated and sits there in a form-fitting and force-locking manner. It serves to provide an attachment point for restraints (tension belts, struts, shells, etc.). The restraints act mutually on one or more sleeves/straps/shells/cups in the lumbar area of the spine and contribute in particular toward partially relieving the stress on the anatomical structures of the spine (lumbar spine) and/or guiding the alignment of the spine and holding it there in the sense of a thoracolumbar orthotic or a lumbar orthotic. Other structural applications include anchoring of plate spring structures which grip the pelvis from the rear for example, where they serve as the basis and anchoring means for back pad supports.

### Example 8

### Leg Orthotic Secured on the Ischial Tuberosity

An appliance like an elastomer ring is provided for the ischial tuberosity. It is covered with an optionally additionally fenestrated rigid shell sitting there in a form-fitting and force-locking manner. The rigid shell serves to provide a point of attachment for the so-called seating in the leg orthotics which are thereby relieved. These act mutually on the ring-like gripping structure, in particular by means of a shaft or a sleeve/strap/shell on a load-bearing orthotic and contribute toward transferring the load of the torso onto the leg orthotic and/or, in the opposite direction, transferring the ground reaction forces of the leg orthotic to the pelvis, and then secondarily transferring the load onto the anatomical structures connected thereto.

### Example 9

### Torso Orthotic Secured on the Trochanter Major

An elastomer ring gripping the trochanter major of the femur is implemented on the femur proximally to the hip. This is covered with an optionally fenestrated rigid shell, which sits there in a form-fitting and force-locking manner. This rigid shell serves to offer an attachment point for restraints (tension belts, struts, shells, etc.). The restraints preferably grip mutually on a sleeve/strap/shell of a torso orthotic appliance and contribute toward guiding the orthotic in its spatial position in relation to the trochanter and/or guiding the ball joint of the femur in the socket and holding it there (hip joint relieving and/or guiding orthotic).

### Example 10

### Knee Brace or Below-the-Knee Brace Secured on the Condylus Medialis

An elastomer ring gripping the condylus medialis of the femur is implemented on the femur near the knee. It is covered with an optionally fenestrated rigid shell which sits there in a form-fitting and force-locking manner. The rigid shell serves to provide a point of attachment for restraints (tension belts, struts, shells, etc.). The restraints preferably act mutually on a sleeve/strap/shell of a knee brace or below-the-knee brace and contribute toward guiding it in its spatial position in relation to the knee joint and/or guiding the knee joint in its articular surface and holding it there (knee-joint-relieving and/or guiding orthotic).

### Example 11

### Knee Brace or Below-the-Knee Brace Secured on the Head of the Fibula

An elastomer ring gripping the caput fibulae is implemented near the knee on the lower leg. It is covered with an optionally fenestrated rigid shell sitting there in a form-fitting and force-locking manner. The rigid shell may offer a point of attachment for restraints (tension belts, struts, shells, etc.). Such restraints preferably act mutually on a sleeve/strap/shell of a knee brace or below-the-knee brace and contribute toward guiding it in its spatial position in relation to the knee joint and/or guiding the knee joint in its articular surface and holding it there (knee-joint-relieving and/or guiding orthotic).

### Example 12

### Malleolar Orthotic Secured on the Malleolus Medialis

An elastomer ring gripping one or both ankle bones, namely the malleolus medialis and lateralis, is implemented on the lower leg proximally to the foot. These are each covered with an optionally fenestrated rigid shell sitting there in a form-fitting and force-locking manner. These rigid shells serve to provide an attachment point for restraints (tension belts, struts, shells, etc.). These restraints may act mutually on a sleeve/strap/shell in the area of the foot and contribute toward guiding the foot in its spatial position in relation to the lower leg and/or the ankle joint in its articular surface and holding it there under the influence of external forces (malleolar guiding orthotic).

### Example 13

### Dorsiflexion Splint Secured on the Metatarsal Bone

An elastomer ring gripping the roughness [sic] of the fifth metatarsal bone is implemented on the foot. It is covered by an optionally fenestrated rigid shell sitting there in a form-fitting and force-locking manner. The rigid shell serves to correct deformities of the foot in another location on the foot or in the area of the fifth metatarsal bone with the remainder of the shell (in this case: orthopedic insert). The rigid shell may be used to provide a point of attachment for restraints (tension belts, struts, shells, etc.). Such restraints may act mutually on a sleeve/strap/shell in the area of the lower leg and may contribute toward guiding the foot in its spatial relationship to the lower leg or it may guide the foot in the knuckle joint and keep it under the influence of external forces there (dorsiflexion splint).

### Example 14

### Quengel Toe Orthotic Secured to the Toe Joint

An elastomer ring gripping the toe joint knuckle is designed for the knuckles of the toe joint and is covered with an optionally fenestrated rigid shell sitting on the knuckle in a form-fitting and force-locking manner. Restraints (tension belts, struts, shells, etc.) are provided with a point of attachment by the rigid shell. Restraints act mutually on the underside of the toes and contribute toward extending contracted joints and/or guiding the toe joints in their articular axis and articular surfaces and holding them there (Quengel toe orthotic, relieving and/or guiding orthotic).

### Example 15

### Knee Brace Secured on the Patella

The patella on a traditional soft good orthotic (for example, SofTecGenu, Bauerfeind company) is enclosed by an oval elastomer ring. Proximally the patella itself is placed in the oval hole-shaped ring opening in the elastomer ring.

The elastomer ring cannot slip off the patella because the ring-shaped opening surrounds the patella on all sides without any tolerance and the flexible textile fabric additionally presses the elastomer ring against the knee in this position (while retaining the required play in movement of the patella).

The oval elastomer ring experiences additional contact pressure due to the rigid shell which is applied from the outside frontally using fastening means according to the invention (with belt-like closures, not shown in the Figure).

The externally applied rigid shell orthotic is in turn held distally in position by the oval elastomer ring which it surrounds and presses through an oval ring-shaped opening in the frontal rigid shell. The oval ring-shaped patellar opening in the rigid shell may be simply cut in a ring shape and without any three-dimensional contouring of the cut edge or it may partially surround the radius of the elastomer ring in a form-fitting manner. The form-fitting grip increases the dimensional stability of the frame shell and reinforces it additionally against (torsional) stress otherwise.

The oval elastomer ring thus forms the following bilateral anchoring of the orthotic:

proximally surrounding the patella

proximally optionally by the coefficient of static friction of the elastomer used

distally in the oval ring-shaped patellar opening in the attached rigid shell

### Example 16

### Knee Brace Secured on the Head of the Tibia

For prevention or treatment of a patellar lateralization (luxation tendency) an improved knee brace is used in which migration of the orthotic on the knee is prevented by an additional enclosure of the tibia head (caput tibiae).

The rigid frame element has a first inventive recess to receive an elastomer coupling element in particular which is positioned over the tibia head as the anatomical protrusion to enable fixation of the orthotic. At the same time the orthotic has at least one additional recess on the same rigid frame part or another rigid frame part which enables therapeutic fixation of the patella and the knee joint via an additional elastomer coupling element which is arranged around the patella. The required contact pressure for securing the rigid frame element over the coupling element to the tibia head is accomplished by belts on the back.

1. An orthotic appliance comprising at least one rigid frame element and at least one coupling element, the rigid frame element having at least one recess to receive the coupling element, such that the rigid frame element can be brought into force-locking engagement over the recess and the coupling element with at least one anatomical protrusion on a human or animal body in order to secure the orthotic appliance in a stationary position on the body.

2. The orthotic appliance according to claim 1, wherein the coupling element is specially designed to extend around the body protrusion in a ring shape, half-ring shape or bracket shape.

3. The orthotic appliance according to claim 1, wherein the coupling element consists essentially of an elastomer material.

4. The orthotic appliance according to claim 1, wherein the recess in the rigid frame element to receive the coupling element is designed as a concave indentation which partially surrounds the circumference of the coupling element.

5. The orthotic appliance according to claim 1, wherein the rigid frame element has a fenestration in the area of the recess.

6. The orthotic appliance according to claim 5, wherein the rigid frame element has an impact protection shell coupled to the fenestration in the area thereof.

7. The orthotic appliance according to claim 1, wherein the rigid frame element is connected to a proximal orthopedic soft good appliance.

8. The orthotic appliance according to claim 7, wherein the in-side of the rigid frame element directed toward the orthopedic soft good appliance is adapted to at least one contact surface section on the surface shape of the outside of the orthopedic soft good appliance directed toward the rigid frame element and a form-fitting coupling of the rigid frame element and the orthopedic soft good appliance can be achieved at least on the contact surface section.

9. A hybrid orthotic appliance comprising an orthopedic soft good appliance, at least one rigid frame element arranged there-on and at least one coupling element.

10. The hybrid orthotic appliance according to claim 9, wherein the coupling element forms a unit together with the orthopedic soft good appliance.

11. The hybrid orthotic appliance according to claim 9, wherein the rigid frame element on the soft good orthotic appliance can be upgraded by means of a detachable force-locking connection comprising a belt connection, a Velcro-type connection, a screw connection, a pin connection or a button connection.

12. A method of supporting a joint, comprising using a ring-shaped, half-ring-shaped or bracket-shaped coupling element in combination with a rigid frame element of an orthotic appliance for securing the orthotic appliance in a stationary position on the human or animal body, wherein the coupling element surrounds an anatomical protrusion on the body in a force-locking manner and the rigid frame element has a recess to receive the coupling element.
